(19) United States
(12) Patent Application Publication
(10) Pub. No.: US 2003/0129267 A1
(43) Pub. Date: Jul. 10, 2003

(54) 100% Kona coffee & honey face mask and skin scrab

(76) Inventor: Michael Lawrence, Bountiful, UT (US)

Correspondence Address: Michael Lawrence, 1390 South, 325 East, Bountiful, UT, 84010, US

(21) Appl. No.: 10/044,033

(22) Filed: Jan. 10, 2002

(51) Int. Cl. A61K 35/78; A61K 35/64

(52) U.S. Cl. 424/776; 424/539; 514/53

(57) ABSTRACT

A unique process of precisely roasting and texture 100% Kona coffee added immediately to pure honey which has been slowly heated to an exact temperature combine to produce a skin cleanser and facial mask which can help to smooth and soften skin. The resulting products a combination of ancient techniques, new technology, and individual inventiveness and ingenuity. The end product is entirely natural and unique.

### CROSS-REFERENCE TO RELATED APPLICATION

Antioxitive Activities of Heterocyclic Compounds Formed in brewed coffee: Marie Deloers Fuster, Alyson E. Micthell, Hirotomo Ochi, and Takayuki Shibamoto. Department of Enviromental Toxicololgy, University of California, Davis, And Japan Institude for the control of Aging, Journal of Agricultural and Food And chemistry. Volume 48, Number 11, Pages 5600-5603

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF INVENTION

The inventor, Michael Lawrence Roasts coffee and started doing research on fresh roasted coffee and its healthy benifits. A cross reference to the ancient Japanese practice of scrubbing skin with coffee grounds. Simultaneously, a friend mentioned to him the skin softening properties of honey. Curious, he experimented with combing the two. After months of trials at different temperatures, using different types of coffee, honey, and equipment, he found the combination herein described. After asking many acquaintance to field test the skin care product for him, he became convinced the product was helpful for many conditions. He began searching for other similar products and found none. Hence, this patent application, and the hope of making the all natural product available to many people at modest prices through effective marketing.

### DRAWINGS

Not applicable

### ABSTRACT AND SUMMARY OF INVENTION

A unique process of precisely roasting and texture 100% Kona coffee added immediately to pure honey which has been slowly heated to an exact temperature combine to produce a skin cleanser and facial mask which can help to smooth and soften skin. The resulting product is a combination of ancient techniques, in technology, and individual inventiveness and ingenuity. The end product is entirely natural and unique.

1. The use of exact ingredients and precise temperatures combined in highly specific ways, captures valuable natural components of the ingredients which have a unique and synergistic effect Upon the skin of the user.
a. 100% Kona Coffee
i Ph of 7.0
ii. Volatile heterocyclic compounds in the Kona coffee help defeat signs of aging when finally ground to a consistency of espresso and combined with honey within 20 minutes of roasting at a temperature range of 356 F. To 376 F.
iii Fine, gritty texture helps exfoliate dead skin cells thereby smoothing and softening the skin.
b. Pure Honey
i Pure honey heated to precisely 205 F. holds the ground 100% kona coffee in suspension as it cool, acting to preserve the Voltile Heterocyclic compounds.
ii Pure honey helps to soften and tighten skin and has a film forming effect holding Valuable valotile heterocyclic compounds left on the skin after rinsing
